(19) United States
(12) Patent Application Publication
(10) Pub. No.: US 2026/0041510 A1
(43) Pub. Date: Feb. 12, 2026

(54) SYSTEM AND METHOD FOR A HYDRAULIC TRANSMISSION SYSTEM OF TELEOPERATION DEVICE WITH HAPTIC FEEDBACK

(71) Applicant: Medical Devices Corner, Inc., Menlo Park, CA (US)

(72) Inventors: Samuel Frishman, Menlo Park, CA (US); John Peter Whitney

(21) Appl. No.: 19/122,280

(22) PCT Filed: Oct. 18, 2023
(86) PCT No.: PCT/US2023/077224

§ 371 Date: Apr. 18, 2025

Related U.S. Application Data
(60) Provisional application No. 63/417,100, filed on Oct. 18, 2022.

(51) Int. Cl. A61B 34/35; A61B 5/00; A61B 5/055; A61B 34/00; B25J 3/04

(52) U.S. Cl. CPC A61B 34/35; A61B 5/0036; A61B 5/055; A61B 34/76; B25J 3/04

(57) ABSTRACT

A system and method for a hydraulic transmission system of a teleoperation device with haptic feedback to be used with a medical scanner can include an input manipulator system situated in a shielded region outside of the medical scanner; an output manipulator system situated within a scanning region of the medical scanner; a hydraulic transmission system comprising: an intermediary active transmission interface serially integrated between the input manipulator system and the output manipulator system and within the shielded region, the active transmission interface comprising a powered actuation system, an output transmission interface that is coupled to the output manipulator system, and a set of output hydraulic lines connecting to the intermediary active transmission interface in the shielded region to the output transmission interface in the scanning region.

### CROSS-REFERENCE TO RELATED APPLICATIONS

This application claims the benefit of U.S. Provisional Application No. 63/417,100 filed on 18 Oct. 2022, which is incorporated in its entirety by this reference.

### TECHNICAL FIELD

This invention relates generally to the field of teleoperated medical devices and more specifically to a new and useful system and method for a hydraulic transmission system of teleoperation device with haptic feedback.

### BACKGROUND OF THE INVENTION

Imaging technology has many benefits in the medical space. However, such advanced imaging is often removed from the actual procedures performed by a doctor. In particular, MRI (Magnetic Resonance Imaging) technology cannot be readily used while electronics or metal devices are in vicinity of the patient. Similarly, CT (Computed Tomography) generates potentially harmful radiation, making it preferable for physicians to be distanced from the scanner when operational. These have been a practical limitation forcing MRIs and CTs to be most typically used separate from actual interventional procedures.

Thus, there is a need in the teleoperated medical device field to create a new and useful system and method for a hydraulic transmission system of teleoperation device with haptic feedback. This invention provides such a new and useful system and method.

### BRIEF DESCRIPTION OF DRAWINGS

FIG. 1 is a schematic representation of one generalized system variation.

FIG. 2 is a schematic representation of a system variation with a single segment of hydraulic lines extending to an output manipulator.

FIG. 3 is a schematic representation of a system variation with two segments of hydraulic lines and an intermediary active transmission interface.

FIG. 4 is a schematic representation of an exemplary system variation showing more than one input manipulator systems.

FIG. 5 is a detailed schematic representation of a system variation.

FIG. 6 is a detailed schematic representation of transmission interfaces used for one channel of a hydraulic transmission system variation.

FIG. 7 is a detailed schematic representation of a system variation with two segments of hydraulic lines.

FIGS. 8A-8C are detailed schematic representations of transmission interface variations used for one channel of a hydraulic transmission system variation.

FIGS. 9A and 9B are detailed representations of a channel of interconnected exemplary transmission interfaces using hydraulic-to-mechanical transmission mechanisms.

FIGS. 10A and 10B are schematic diagrams of exemplary implementation of the system.

FIG. 11 is a detailed schematic representation of a system variation with a digital control input and control system.

FIG. 12 is a detailed schematic representation of a system variation with a data input and control system.

FIG. 13 is a schematic diagram of an exemplary implementation showing hydraulic subsystems.

FIGS. 14A and 14B are schematic representations of system variations used across two regions.

FIG. 15 is a schematic representation of control system independently controlling subsets of channels of a hydraulic transmission system.

FIGS. 16-18 are flowchart representations of method variations.

FIG. 19 is an exemplary system architecture that may be used in implementing the system and/or method.

### DETAILED DESCRIPTION OF THE EMBODIMENTS

The following description of the embodiments of the invention is not intended to limit the invention to these embodiments but rather to enable a person skilled in the art to make and use this invention.

### 1. Overview

Systems and methods for a hydraulic transmission system of a remote haptic interface function to use an integrated active transmission interface that can provide power-assisted actuation and/or sensing capabilities. The systems and methods use a set of hydraulic lines to translate forces and actuation between input and output manipulators. As an intermediary component between the input and output, an active transmission interface can be used to augment the hydraulic system with dynamic augmentation of actuation and/or forces transmitted within the hydraulic lines and/or with added sensing capabilities.

In some variations, the systems and methods may include a human controlled input manipulator system (e.g., a mechanical controller) that is used to control and at least partially drive a remote output manipulator system (e.g., an end effector). The systems and methods may additionally enable transmission of haptic feedback from the output to the input manipulator, which may function to provide responsive tactile haptic feedback to the operator of the input manipulator system. The systems and methods may be used to enable remote operation of some manipulator device that can be used in medical procedures or other areas of use.

The systems and methods are specially configured to enable the combination of an active electronic-based system while being configured such that material requirements for the different components enable it to be used in situations suitable for operation of a manipulator in restricted environments such as alongside medical scanners like an MRI machine. As an active electronic-based system the systems and method may benefit from active motor control, digital control of such motors, and/or digital sensing. The special configuration and use of hydraulic actuation is used to allow spatial segregation of materials based on the environment of use. This may make the systems and methods compatible for use alongside MRI (Magnetic Resonance Imaging) devices, CT (Computed Tomography) devices, and/or other sensitive devices. For example, the system's and method's approach to integrating electronic components can enable the distal end of the device (e.g., the output manipulator and the output hydraulic lines) to be made of MRI compatible materials. The non-compatible materials may be integrated within components that can be positioned out of range, shielded, or otherwise safe from the sensitive device(s). As such, the device may be used for operating on a patient while the patient is being imaged in an MRI machine.

In particular, the systems and methods use a hydraulic transmission system with an active transmission interface that couples two haptic manipulators. The active transmission interface in one variation includes a powered actuation system (e.g., an electric motor). The powered actuation system can be configured within the hydraulic transmission system such that it can intervene to actively augment the actuation output and/or haptic feedback and actuation (i.e., forces) that are translated (bidirectionally) between input and output manipulators.

A powered active transmission interface may be used to correct or adapt haptics. This may, for example, be used to increase or decrease the resistive force felt by an operator using the input manipulator system. In some cases, this may be used to counteract resistive forces resulting from the hydraulic system and/or other mechanisms used in the system to provide a more natural or realistic translation of haptic feedback from the output to the input.

The systems and methods can additionally enable dynamic changing of control modes. The configuration of the systems and methods can enable a range of control modes ranging from passive control (where powered active control of the active transmission interface is disabled) to semi-automated (e.g., using combination of operator actuation input and powered actuation) and/or fully automated (e.g., using powered actuation without actuation input from the input manipulator system).

In some variations, the systems and methods may enable the device to be manipulated fully manually (i.e., passive mode), with power assist (e.g., using powered actuation within the active transmission interface), and/or in a fully autonomous mode, all within the same device. A device's ability to have fully manual and fully automated modes within the same device may provide a number of usability and reliability benefits.

In some variations, the active transmission interface may additionally or alternatively include a sensing system that is used to monitor and sense the actuation and state of the system. A sensing system may be integrated to create a sensor-enabled active transmission interface. It may also be integrated in combination with a power-assisted variation to enable a powered and sensor-enabled active transmission interface. A sensor-enabled active transmission interface may be used to monitor the state of the device. This may also be used to collect data on the use of the device during different variations, which could be used in enabling data-driven dynamic control of the device.

Herein, the transmission system with powered and/or sensing capabilities is primarily described as an intermediary subsystem oriented between the input and the output. In some variations, a powered actuator variation or sensing variation of the transmission interface may be configured at an input side of the hydraulic transmission system. Configuration of the system to have an intermediary transmission interface away from the output may enable better actuation control and/or sensing, while keeping the output manipulator composed of materials and elements that can be used within the bore of an MRI machine.

The systems and methods may further include components that enable a highly flexible and versatile system that can be used in a variety of ways. For example, the systems and methods may be used for enabling remote operation of a manipulator with one or more degrees of freedom. When there is a plurality of degrees of freedom within a manipulator, the different degrees of freedom may be controlled individually or in groups. For example, a given subset of degrees of freedom may be locked, made to operate passively, or to have any form of semi- or fully-automated modes of control. Additionally, the systems and methods may include design features that enable reconfiguration of the system so that it can be adapted to different designs of manipulators or different uses. With such flexibility and modularity, the systems and methods may be highly adaptable for use in a medical setting. The various manipulators, hydraulic lines, and transmission systems may be configured so that they can be disconnected and reconnected without substantially loss of pressure to the hydraulic system. This may be used to quickly prepare or clean up after a procedure. For example, some components could be swapped out between procedures for sanitation reasons. The modular configuration may also enable the device to be customized for different usages. For example, the device could be reconfigured to be a fully passive system (e.g., without an active transmission interface), to include a different output manipulator (e.g., end effectors), and/or to use a different input manipulator.

The systems and methods are preferably used in enabling a device that can be used for performing medical procedures. In one particular application, the systems and methods may be used for performing procedures on a patient with live MRI monitoring of the patient. The output side of the device can be made free of metals or electronics such that it can be used on a patient within the bore of an MRI machine. The human operator and the active transmission interface can be positioned away from the MRI machine. In some cases, the input manipulator system and the active transmission interface and/or other components (that are not compatible with use near a scanning device) can be located in a shielded environment. The hydraulic lines can be the mechanism through which remote actuation control is transported. The device may additionally or alternatively be used in combination with other imaging technologies such as CT scanners, ultrasound imaging, or other imaging technologies. The systems and methods are not limited to be using in combination with such imaging technologies. The systems and methods provide numerous advantages such as enhanced haptic feedback, dynamic control features, and/or possibility for direct human mechanical control, which have advantages over other offerings even without using the device in a material-restricted application. The ability to perform procedures simultaneously with advanced imaging can enable novel user interfaces such as providing AR/VR user interfaces for use while using the system and method.

The systems and methods may be adapted for any suitable types of input control and output manipulation. In one variation, the systems and methods are used to enable performing biopsies and ablations, heart procedures, neurosurgical operations, and/or other operations or procedures.

Herein, the systems and methods are described primarily as having an input portion of the device (the portion used by one operator) and an output portion of the device (the portion performing the intended actuation). The systems and methods may alternatively be adapted for having multiple inputs (used by multiple people) and/or having multiple manipulators (multiple distinct end effectors).

Additionally, in some variations, the system may be used in a context where there is less distinction of an input and output, and instead actions/feedback are transmitted between two or more manipulators bidirectionally. Accordingly, herein, references to input and output are used to characterize general roles of different portions of the system and method for convenience. Actuation may be bidirectional such that supplied direction or force input may be supplied from either input or output and similarly affected or delivered force may be delivered to either input or output. As such input or output may alternatively be characterized with alternative labels such as first and second or controller and end effector.

The system and method may provide a number of potential benefits. The system and method are not limited to always providing such benefits and are presented only as exemplary representations for how the system and method may be put to use. The list of benefits is not intended to be exhaustive and other benefits may additionally or alternatively exist.

As one potential benefit, the systems and methods may enable teleoperation of a medical surgical/operation tool with live imaging. As discussed, the systems and methods can eliminate incompatible materials and components from the output manipulator such that the device can be used, for example, inside of the bore of an MRI machine. Physical operation on a patient may then be performed while live MRI imaging is performed. This could similarly be applied to other types of imaging. This could result in procedures being more successful and/or being performed in less invasive ways.

As another potential benefit, the systems and methods may provide a solution that offers realistic and useful haptic feedback. The hydraulic system can preferably translate haptic feedback from the output manipulator back to the input manipulator. This can be important to providing a doctor with enhanced control while performing a procedure. Furthermore, a powered active transmission interface may be used to correct for resistance or non-linearities in the hydraulic transmission such that haptic feedback is controlled. This may be used to provide more of a more realistic feeling to actions and tactile feedback. This may additionally or alternatively be used for synthetically altering the haptic feedback based on various conditions. For example, synthetic walls or barriers could be simulated by the device to prevent actuating into restricted areas.

As another potential benefit, the device of the systems and methods may be operated in various modes, ranging from a purely passive mode (primarily using the hydraulic transmission system to passively drive the output) to a fully autonomous mode, where the powered active transmission interface is used to drive the output. Power-assist modes may also be used to provide features like removing tremor from the user input, preventing or restricting potentially unintended inputs (e.g., sudden jerking motions), translating input motions to a different output motion (e.g., scaling up or down amount of movement), or for other operations. The systems and methods may additionally dynamically alter modes of operation. For example, the systems and methods may switch between different control modes, enable multiple modes simultaneously, use different modes for different degrees or freedom, and/or dynamic shifting of modes. These modes and/or the changing of modes may be manually or dynamically enabled and disabled.

As another potential benefit, the systems and methods may have actions of the device tracked using an integrated sensing system. Data can be collected as an operator uses the device. This may be used to track, monitor, and/or model actions when performing a procedure. Collected data may be used to customize the operation of a device for a user. The collected data may additionally or alternatively be used in enabling automated control of the output.

As another potential benefit, the systems and methods may be a modular and easily configured device. This configurability of the device may provide many affordances helpful when used in a medical setting. For example, the systems and methods may have swappable end effectors for fast setup and breakdown before a procedure. In some variations, the systems and methods may make some components using cost effective manufacturing techniques and materials so that the system components could be disposable or more easily washed for meeting the sanitation challenges of a medical setting.

### 2. System

A system for a hydraulic transmission system of a teleoperation device with haptic feedback may include a hydraulic transmission system that can interface with at least two manipulator systems. The hydraulic transmission system preferably connects to and transmits actuation/haptic control between an input manipulator system and the output manipulator system. In some variations, the system may include the manipulator system(s). In some variations, the system may include interfaces to engage with a compatible external manipulator system (e.g., where the system is provided without an external manipulator system). Herein, the system is described including description of the external manipulator system, but the system is not limited to always including such a component.

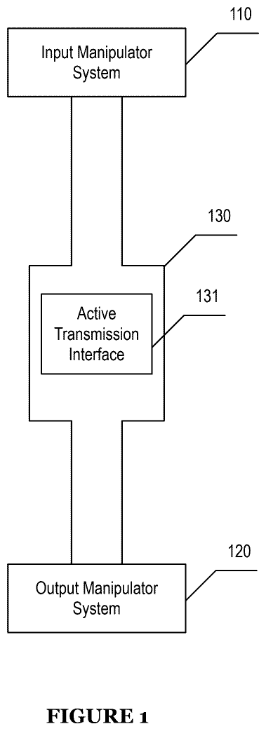

In one variation shown in FIG. 1, the system may include an input manipulator system 110, an output manipulator system 120, and a hydraulic transmission system 130. The hydraulic transmission system 130 preferably couples actuation of the input manipulator system 110 and the output manipulator system 120 at least partially through at least one hydraulic line. The hydraulic transmission system 130 preferably includes an intermediary active transmission interface 131 serially integrated with the at least one hydraulic line. The active transmission interface 131 can enable augmentation of actuation and/or sensing of actuation.

The system uses multiple “interfaces” between hydraulic systems, mechanical systems, active motor controlled and/or sensored systems, and/or digital/computer controlled systems. These components are integrated to enable enhanced capabilities at the distal “output” manipulator that traditionally are not possible within restricted environments such as inside of a bore of an MRI machine during use. The output portion of the system is preferably used to extend into a restricted region, and the materials used on the system components in this region may be limited to compatible materials. The restricted region in one potential application can be a region where a scanner is being used. The other components may be positioned or housed in a region without the restrictions (e.g., a shielded environment). Use of electronics and other materials may be used for components in the shielded/unrestricted environment. The use of hydraulics as a mechanism for conveying actuation is used to bridge the distance between these environments, enabling segregation of components.

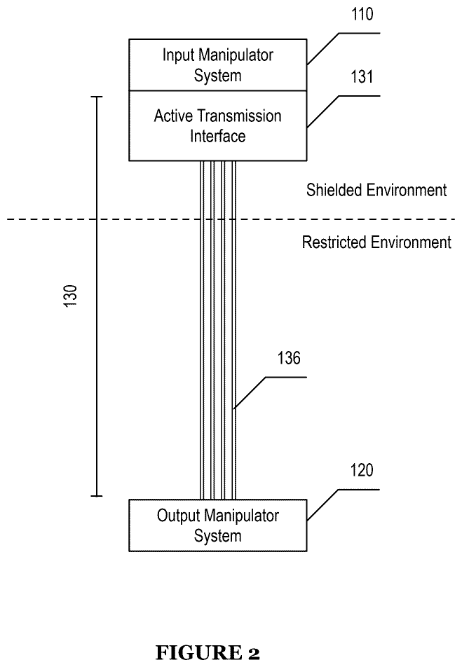

The topology or network of interconnections of the system may be configured in a variety of ways. As shown in FIG. 2, in one variation, a set of output hydraulic lines 136 are used to couple actuation output from the active transmission interface 131 to an output manipulator system 110. The active transmission interface 132 may be coupled to an input manipulator system 110 in some manner.

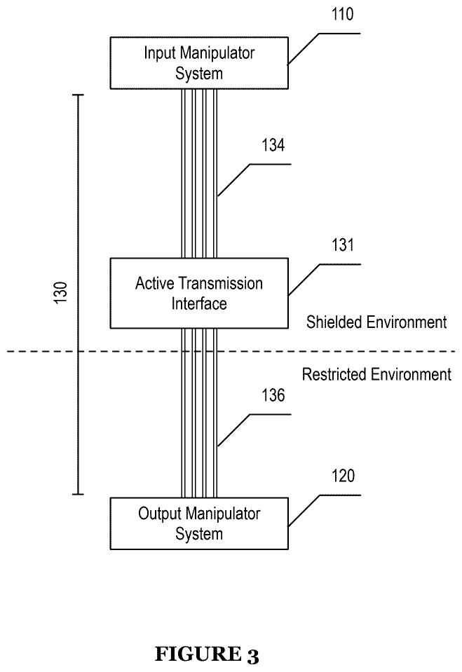

Alternative networks of interconnections may alternatively be used. As shown in FIG. 3, in another variation with two legs of hydraulic lines, a set of input hydraulic lines 134 may be used to couple actuation between the input manipulator system 110 and the active transmission interface 131, and a set of output hydraulic lines 136 may be used to couple actuation between the active transmission interface 131 and the output manipulator system 120. This variation may function to enable a symmetrical system.

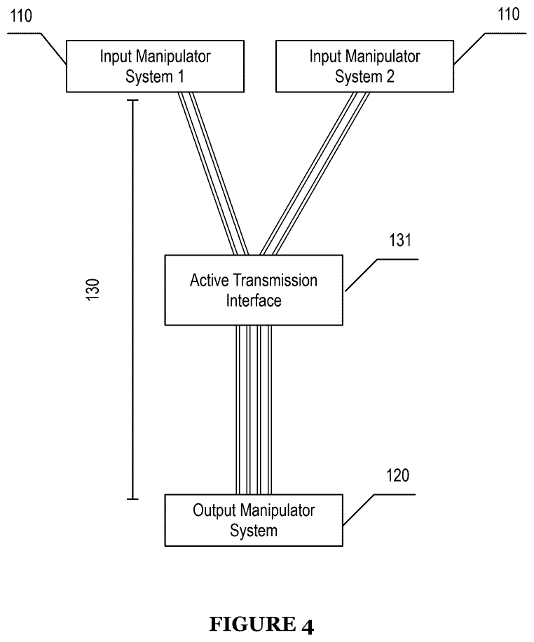

The systems are described herein primarily as including an input and an output. As discussed, the roles of these components may not strictly always be input of instructions and output as in the resulting action. Actuation and forces can be bidirectionally applied such that forces at the input are translated to the output and similarly forces at the output may be translated to the input. Additionally, the system is not limited to only having a single input and/or output. There may be multiple input manipulator systems 110 and/or multiple output manipulator systems 120. As shown in the example of FIG. 4, one variation may include two distinct input manipulator systems 110. The two input manipulator systems may be used to enable different aspects of the output to be controlled by two distinct users. Herein, the examples primarily describe a topology of a single manipulator input to a single output manipulator, but any suitable topology of inputs and outputs may be configured.

In one generalized variation, the system can include an input manipulator system 110; an output manipulator system 120; a hydraulic transmission system 130 comprising: an intermediary active transmission interface 131 serially integrated between the input manipulator system 110 and the output manipulator system 120, the active transmission interface 131 comprising a powered actuation system 132, an output transmission interface 137 that is coupled to the output manipulator system 120, and a set of output hydraulic lines 136 that connect on one end to the active transmission interface 131 and on the opposing end to the output transmission interface 137.

The system is preferably capable of having the active transmission system 131 being used to passively transfer actuation through it without intentional and active intervention/augmentation by a powered actuation system 132. Accordingly, force applied to the input manipulator system 110 or the output manipulator 120 is mechanically transferred through the active transmission interface with optional intervention (e.g., augmentation or autonomous control) by the powered actuation system. The intervention may be active augmentation or autonomous control whereby motors or other actuation systems are actively powered and driven for applying some active change to forces (e.g., actuation) transferred through the active transmission interface 131. The system may be used to transfer and augment actuation and force from input to output and/or from output to input. Accordingly, input applied to the input manipulator 110 is mechanically reflected in the output manipulator 120 via the active transmission interface 131 with or without intervention (e.g., active augmentation) by the powered actuation system 132. Similarly, input applied to the output manipulator 120 is mechanically reflected in the input manipulator 110 via the active transmission interface 131 with or without intervention (e.g., active augmentation) by the powered actuation system 132.

In addition to or as an alternative to the powered actuation system 132, the system may include a sensing system 133 coupled or integrated with the active transmission interface 131 (or the powered actuation system 132). The sensing system 133 can be configured to measure actuation transmitted through the hydraulic transmission system.

The system can additionally include a control system 140 that includes one or more computer processors and computer readable mediums (e.g., non-transitory computer readable mediums) storing instructions that when executed by the one or more computer processors cause the control system to perform various operations. These operations may be used in controlling the powered actuation system 132, receiving data from the sensing system 133, and/or interacting with other system components or external systems. The control system 140 can include configuration to control the powered actuation system 132 to at least partially augment hydraulic actuation/haptic interactions translated through the active transmission interface 130 between the input manipulator system 110 and the output manipulator system 120.

The system may be configured for one or more degrees of freedom. Each degree of freedom may be supported by having a distinct pathway coupling a mechanical degree of freedom of an input manipulator system 110 to a degree of freedom of the output manipulator system 120. Each degree of freedom accordingly can include a distinct “channel” (or pathway) made up of series of interconnected transmission interfaces (e.g., active transmission interface, output transmission interface and optionally input transmission interface) and distinct segments of hydraulic lines. Each degree of freedom may additionally include an active transmission interface instance with a distinct coupling to the powered actuation system 132 (and distinct motor coupled to an active transmission interface) and/or sensing system 133. A shared control system or individual control system may be used in controlling each degree of freedom. Herein, an individual subsystem used to manage one degree of freedom may be referred to as a channel or pathway of the hydraulic transmission system. Each channel may be configured in substantially similar way for a given system implementation. However, the channels of the hydraulic transmission system 130 may be configured with differing designs and features depending on the implementation. The channels may be controlled and augmented individually and independently.

The system in one preferred area of use is with a medical scanner like an MRI device. The system can enable teleoperation of some manipulator (e.g., an articulated syringe, arm, probe, etc.) within the medical scanner even during use. Different components of the system may be situated within an environment according to their function and design. In one variation, the input manipulator system 110 and the intermediary active transmission interface 131 are positioned within a shielded environment (e.g., an unrestricted environment), and the output manipulator system 140, and at least part of the set of output hydraulic lines 136 and the output transmission interface 137 are in a scanning region of a medical scanning device. The output manipulator system 140 in addition to the output transmission interface 137 and at least part of the output hydraulic lines 136 within a restricted region may be made of material compatible with the restricted environment (e.g., compatible with operation of an MRI machine).

Accordingly, in a system for remote operation of a device (e.g., a teleoperated device) within a medical scanner, the system may include an input manipulator system 110 situated in a shielded region (or unrestricted region) outside of the medical scanner; an output manipulator system 120 situated within a scanning region of the medical scanner; and a hydraulic transmission system comprising: an intermediary active transmission interface 131 serially integrated between the input manipulator system 110 and the output manipulator system 120 and within the shielded region, the active transmission interface 131 comprising a powered actuation system 132, an output transmission interface 137 that is coupled to the output manipulator system 120, and a set of output hydraulic lines 136 connecting or coupling to the intermediary active transmission interface 131 in the shielded region and the output transmission interface 137 in the scanning region (e.g., coupling to the output manipulator system 120).

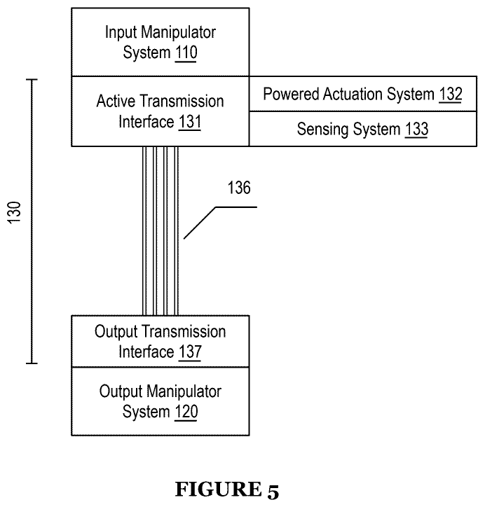

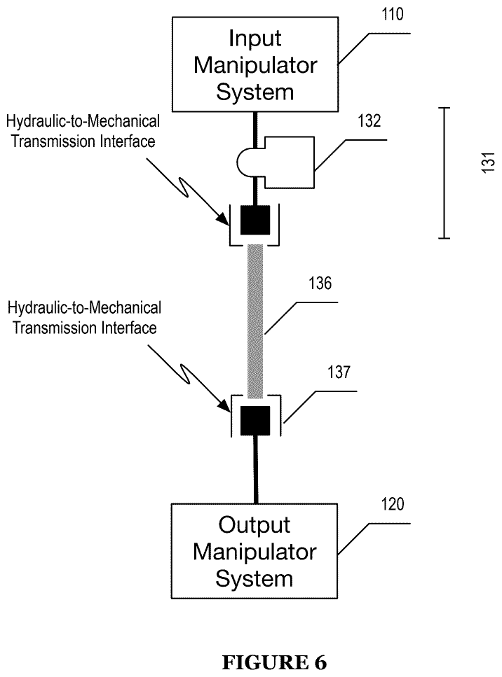

In one system variation, the system may integrate the active transmission interface 131 with a more direct mechanical coupling to the input manipulator system 110. This variation may include a single leg of hydraulic lines in the form of the output hydraulic lines 136 as shown in FIG. 5. The system can include an input manipulator system 110; an output manipulator system 120; a hydraulic transmission system 130 comprising: an intermediary active transmission interface 131 serially integrated between the input manipulator system 110 and the output manipulator system 120, the active transmission interface 131 comprising a powered actuation system 132, an output transmission interface 137 that is coupled to the output manipulator system 120, and a set of output hydraulic lines 136 that connect on one end to the active transmission interface 131 and on the opposing end to the output transmission interface 137, and wherein the input manipulator system is directly mechanically coupled to an input interface of the intermediary active transmission interface. As shown in a FIG. 6, for one channel, the active transmission interface 131 may at an input interface mechanically couple to the input manipulator system (e.g., through a mechanical linkage) and at an output interface couple to the output hydraulic lines (e.g., using a hydraulic-to-mechanical transmission interface to convert between mechanical actuation and hydraulic actuation, such that actuation can be conveyed to/from the output manipulator).

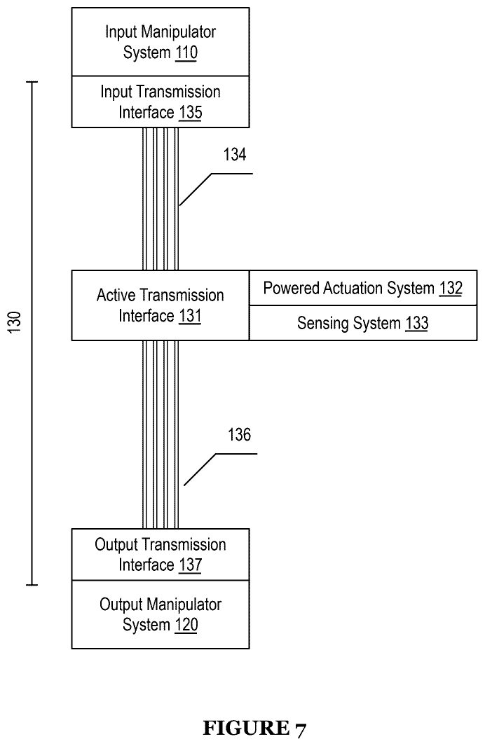

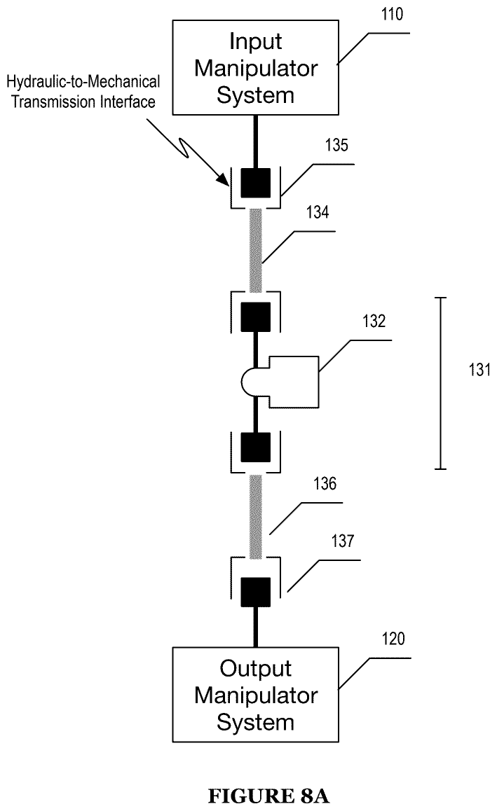

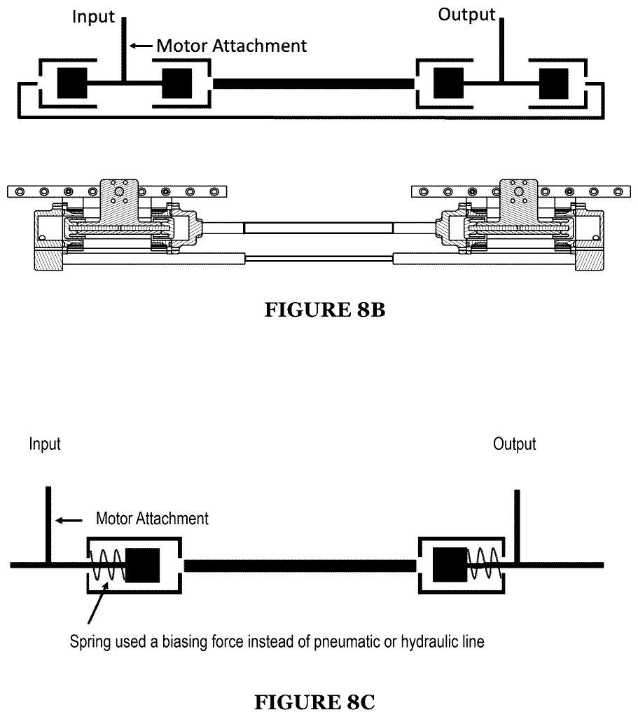

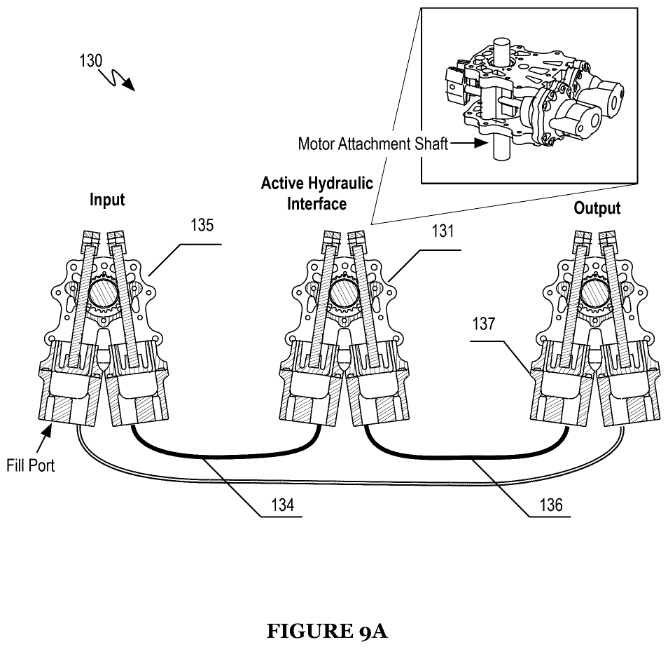

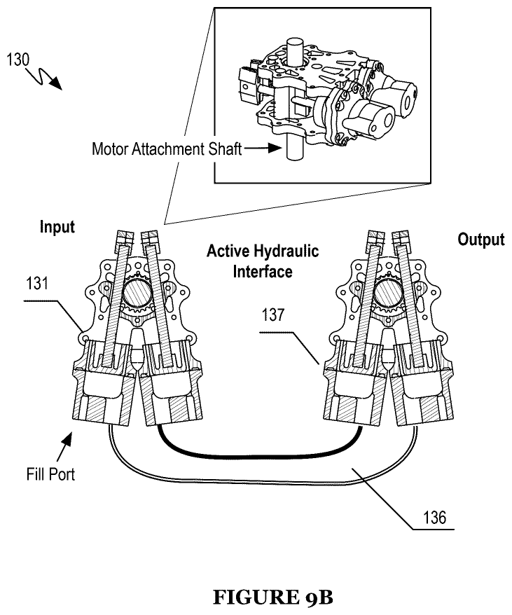

In another system variation, the active transmission interface may be an intermediary component integrated in series within hydraulic lines between the input and output. This variation may include two legs of hydraulic lines with a set of input hydraulic lines and a set of output hydraulic lines as shown in FIG. 7. The system can include an input manipulator system 110; an output manipulator system 120; a hydraulic transmission system 130 comprising: an intermediary active transmission interface 131 serially integrated between the input manipulator system 110 and the output manipulator system 120, the active transmission interface 131 comprising a powered actuation system 132, an input transmission interface 135 that is coupled to the input manipulator system 110 and a set of input hydraulic lines 134 that connect on one end to the active transmission interface 131 (e.g., via a hydraulic-to-mechanical input interface) and on the opposing end to the input transmission interface 135, and an output transmission interface 137 that is coupled to the output manipulator system 120, and a set of output hydraulic lines 136 that connect on one end to the active transmission interface 131 and on the opposing end to the output transmission interface 137. As shown in FIG. 8A, for a single channel, the active transmission interface 131 may at an input interface couple to the input hydraulic lines (e.g., using a hydraulic-to-mechanical transmission interface to convert between mechanical actuation and hydraulic actuation, such that actuation can be conveyed to/from the input manipulator) and at an output interface couple to the output hydraulic lines. As shown in FIGS. 9A and 9B, a set of hydraulic-to-mechanical transmission interfaces may be connected for one channel to facilitate translation between hydraulic actuation and mechanical (e.g., linkage-based) actuation. These transmission interfaces can expose mechanical components that can be used for integrating the powered actuation system 132 and/or the sensing system 133.

As shown in FIG. 8B, a single channel for a system with a single leg of hydraulics, may include two hydraulic lines. In some variations, a pneumatic line may be used in place of one of the hydraulic lines. These may be used to couple two hydraulic interfaces that have a hydraulic-mechanical mechanism that facilitates conversion between hydraulic force and mechanical force. The two lines can act antagonistically. Alternative configurations may also be used. For example, if a restorative force is used within a hydraulic mechanical mechanism, like a spring, then a single hydraulic line may be used. As shown in FIG. 8C, a spring may be used for biasing force instead of a pneumatic or hydraulic line. In this configuration, springs are used for the pressure in the hydraulic line. A motor (of the powered actuation system 132) can be used to compensate for the linear increase/decrease in force from the spring as the piston moves from the central position, turning the springs influence on the system into a constant force, rather than one that changes with displacement.

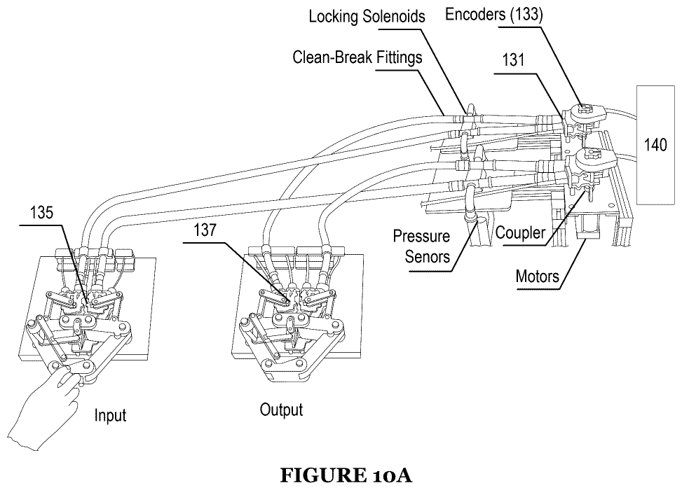

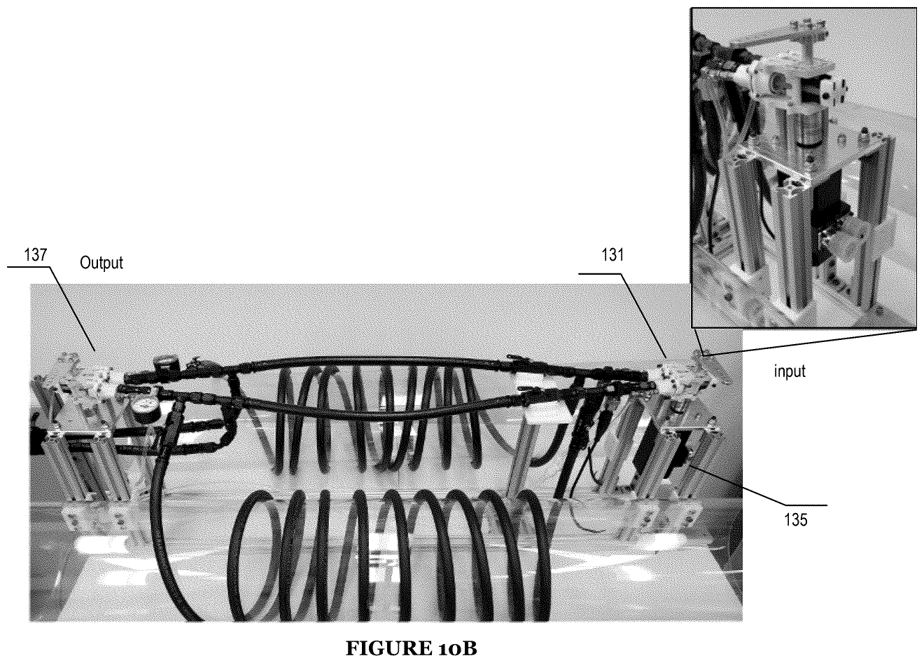

As shown in FIG. 10A and FIG. 10B, an exemplary powered and sensor-enabled transmission interface 131 can be implemented as an intermediary element between an input and output manipulator system. FIG. 10A show illustrative benchtop demonstration of how an input manipulator system 110 and an output manipulator system 120 may be integrated in a symmetrical manner. As shown in FIG. 10A, the system may include an input and output manipulator, integrated through a hydraulic transmission system 130. The hydraulic transmission system may include hydraulic lines with integrated locking solenoids (e.g., usable to lock DoF), clean-break fittings (e.g., for ease of modular adjustments and reconfiguration), motors and couplers used by a powered actuation system 132 to optionally augment or intervene with the actuation of the active transmission interface, pressure sensors, encodes, and a connected control system 140. The control system may integrate motor drivers, power supply, and/or other components used to drive the powered actuation system 132 or any other electrically controlled component.

In a similar variation shown in FIG. 10B, an active transmission interface may be mechanically coupled to an input, which may reduce the hydraulic lines to a single leg of hydraulic transmission.

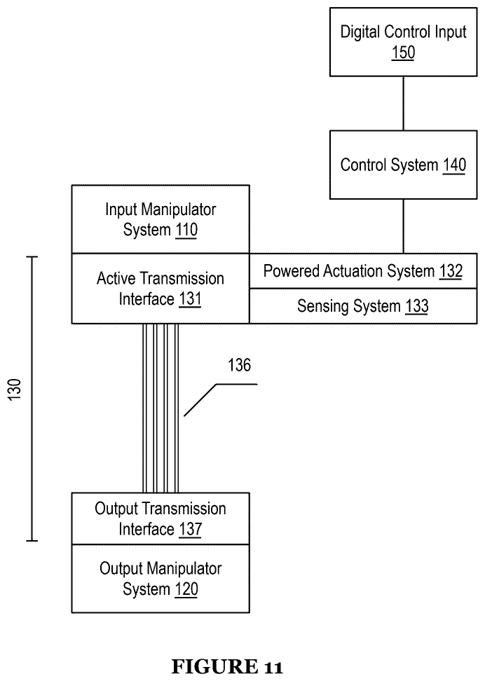

In addition to working with input manipulator systems coupled to the system for direct actuation/haptics, the system may additionally include additional inputs which could be a digital user control input and/or some automated control input. Accordingly, in some variations the system may additionally include a digital control input 150 as shown in FIG. 11. The digital input may be used to dynamically augment or supplement input from the input manipulator system 110. Accordingly, the output of the intermediary active transmission 131 may be based on a combination of the input received from the input manipulator system and the additional digital control input 150. The digital control input 150 may be a communicated digitally to the control system and/or the powered actuation system 132/active transmission interface 131. In some cases, the digital control input 150 may be connected via a network and could be a control input supplied from any network connected device. In some modes, the digital control input 150 may be used to control one or more degrees of freedom and/or all actuation of the output manipulator system 120.

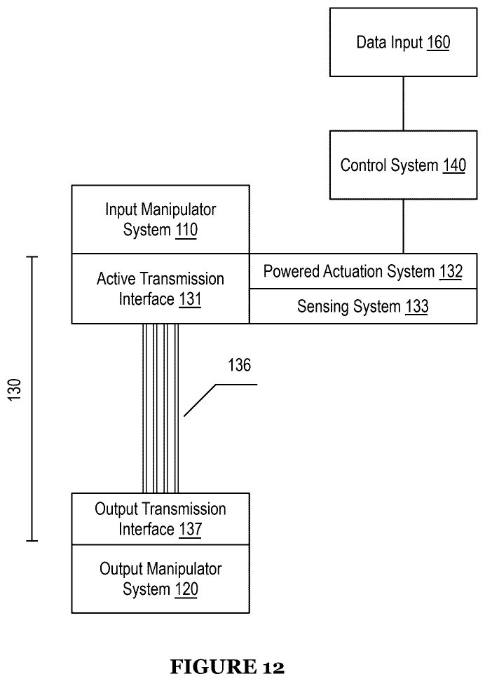

In a similar variation, the system may additionally include data inputs 160 or more specifically one or more types of medical data input as shown in FIG. 12. The control system may control the powered actuation system 132 (or otherwise control the active transmission interface 131) at least partially based on the data inputs. Data inputs 160 may include scanner data from the medical scanner or other sensor data collected and/or processed outside the system (e.g., MRI imaging data, CT scanner data, respiratory rate, heart rate, etc.). The control system 140 may additionally process or analyze the medical data input. The control system 140 may use data from the data input 160 to augment actuation in any suitable manner such as triggering haptic feedback based on the medical data input. For example, the system could simulate increased resistance when a device controlled by the output manipulator system 120 is approaching a detected “out of bounds” region. In another example, the system may high-pass filter or otherwise smooth actuation when in a certain area to avoid any slight tremors or accidental quick motions applied to the input manipulator system 110.

The input manipulator system 110 functions as the component or set of components used by an operator to control the output. The input manipulator systems 110 may be a single system used as a manipulator, which may alternatively be characterized as an actuator, end effector, or input device. The input manipulator system 110 is preferably a haptic controller that has direct physical coupling to the system such that force applied to the input manipulator system translates to force translated through the system and similarly forces be translated through the hydraulic transmission system 130 to the input manipulator system 110 as haptic feedback.

The input manipulator system 110 and similarly the output manipulator 120 may include a set of different levers, pulley systems, sliders, rotators, linkage systems, and/or other mechanisms that can be manipulated and actuated in a way that translates to actuation within the hydraulic transmission system 130.

The input manipulator system 110 can provide a configured set of degrees of freedom (DoF) used to direct actuation input into the system. The manipulator may use various mechanisms and/or linkage systems for offering different DoF. In one variation, the input manipulator system 110 can have six DoF of movement to enable full translational and rotational control of an output manipulator. In some variations, the input manipulator system 110 can include additional input elements with their own DoFs or input mechanisms such that other actions may be performed. For example, input mechanisms may be integrated into the input manipulator so that a needle or probe can be inserted or axially rotated.

Each DoF of the input manipulator system 110 may be coupled directly or indirectly to the hydraulic transmission system 130 and more specifically to the active transmission interface 131. In other words, a dedicated channel of the hydraulic transmission system may interface with each degree of freedom of an input manipulator system 110. In one variation, the input manipulator system 110 is mechanically coupled to an input of the active transmission interface 131. As such there may, in some variations, be no hydraulic actuation on the “input side” of the system. In another variation, the input manipulator system 110 may mechanically couple to a set of input hydraulic lines 134 through an input transmission interface 135. In this variation, the input manipulator system 110 has actuation conveyed through a hydraulic system to the active transmission interface 131, where it may then be conveyed on (possibly in an augmented manner) to the output manipulator system 120.

Haptic feedback is preferably delivered through the mechanical resistance or force applied from the hydraulic transmission system back to the input manipulator system 110.

The controlled DoFs of the input manipulator system 110 are generally described herein as being controlled through manipulation of a mechanical mechanism. In some variations, one or more DoFs may not be a mechanical input but could be fully or optionally controlled electronically. For example, one particular mechanical motion may be controlled using digital control from a computer or an electronic user interface.

The input manipulator may be formed or shaped with ergonomic considerations of the intended user.

In some variations, the input manipulator system 110 is the same, mirrors, or is substantially similar to the output manipulator system 120. However, the form and design of the input manipulator system 110 may vary in any suitable way or even be a distinct design from the output manipulator system 120.

The input manipulator system 110, in some variations, may be used in a less restrictive environment. As such, the input manipulator system 110 may be designed with fewer restrictions/limitations in the materials and types of components used in comparison with the output manipulator system 120.

The output manipulator system 120 functions as the one or more end effector where the intended output actions are performed. The output manipulator system 120, when used in a medical setting, is preferably the component used to perform the actions to a patient.

When the system is to be used alongside imaging technology like MRI, then the output manipulator system 120 may be made of compatible materials such as using plastic or other non-metal components. For example, the output manipulator system 120 may be made of materials compatible with an MRI machine or other type of scanner device. Scanner compatible materials may include non-magnetic materials, non-conductive materials, radio frequency (RF) transparent materials, non-ferromagnetic materials, and/or other material types that would be suitable for use in environments or conditions with material restrictions. This may additionally mean that system components in the restricted environment may lack any electronics.

The output manipulator system 120 may be mounted or positioned within a scanning region of a medical scanning device. For example, during use, the output manipulator system 120 may be oriented within a bore of an operating MRI machine.

Reference to the output manipulator system 120 as an output is used to primarily characterize its general role as the component used to perform a desired action. Force can similarly be conveyed from the output manipulator system 120 back to the hydraulic transmission system 130 and back (possibly in an augmented form) to the input manipulator system 110. Physical feedback imparted onto the output manipulator is preferably translated back as haptic feedback to the input manipulator system 110.

The hydraulic transmission system 130 functions to relay actuation between input manipulator system 110 and the output manipulator system 120. More specifically, the hydraulic transmission system 130 uses a set of hydraulic lines to convey actuation over some distance such that the input manipulator system 110 and the output manipulator system 120 may be separated by some distance. This distance of separation may be of arbitrary length but generally could range from 3 to 20 or more feet. There is preferably at least one hydraulic line for each channel (or DoF).

In one variation, the hydraulic transmission system 130 includes an intermediary active transmission interface 131 between the input and output manipulator systems 110 and 120, with at least one segment of hydraulic lines used to convey actuation. The hydraulic transmission system 130 may include hydraulic lines, hydraulic transmission interfaces, and optionally a powered actuation system 132 and/or a sensing system 133. The hydraulic transmission system 130 can be a serial network of hydraulic lines and transmission interfaces (e.g., hydraulic-to mechanical transmission interfaces), with optionally integrated powered actuation, sensing, and/or control components. The hydraulic transmission system 130 can include multiple channels to support transmission of multiple degrees of freedom.

Reference to a hydraulic transmission system 130 is used to describe a set of integrated subcomponents. The subcomponents of the system may alternatively be separated, grouped, and/or described in any suitable manner, including referencing their interconnected nature without referencing the hydraulic transmission system.

Accordingly, in one variation, the hydraulic transmission system 130 (or the system more generally) may include an intermediary active transmission interface 131 serially integrated between the input manipulator system 110 and the output manipulator system 120, an active transmission interface 131 comprising a powered actuation system 132 and/or a sensing system 133, an output transmission interface 137 that is coupled to the output manipulator system 120, and a set of hydraulic lines. The set of hydraulic lines preferably includes a set of output hydraulic lines 136 connecting to the intermediary active transmission interface 131 and the output transmission interface 137.

When there are two legs of hydraulic lines, the system may additionally include an input transmission interface 135 that is coupled to the input manipulator system, and additionally include a set of input hydraulic lines 134 connecting to the intermediary active transmission interface 131 and the input transmission interface 135. In this way, a hydraulic line may be established between the input and output with the active transmission interface being used to integrate a powered system and/or a sensing system.

The set of hydraulic lines functions to transmit hydraulic power from one component of the system to another. The hydraulic lines can transmit power thereby conveying actuation and/or force through the hydraulic lines. The system can convert pressure of hydraulic fluid in the hydraulic lines to kinetic energy to actuate a mechanical mechanism. The hydraulic lines preferably terminate with a transmission interface (e.g., a hydraulic-to-mechanical transmission system) to convert between hydraulic actuation and mechanical actuation.

The Hydraulic lines may be flexible or rigid. Flexible hydraulic lines may provide flexibility of use when used in a medical setting where equipment may need to be moved and adjusted. Combinations of flexible sections with rigid sections of hydraulic lines may also be used.

There is preferably a hydraulic line for each independent degree of freedom in a connected manipulator system. In some variations, the input manipulator system 110 and the output manipulator system 120 include corresponding numbers of DoF. There can be a hydraulic line for each channel or degree of freedom (e.g., one output hydraulic line and one input hydraulic line).

These distinct lines may make each DoF individually controllable such that each DoF may be controlled in different, independent ways. For example, a first DoF may be set to a passive mode where actuation is not augmented, while a second DoF may be augmented in a way that uses the powered actuation system to alter input from the input manipulator system 110 for the second DoF, a third DoF may be fully automated, and a fourth DoF may be locked to prevent actuation on that DoF. The modes of the different DoFs can also change independently. Accordingly, the control system can control the powered actuation system to augment hydraulic actuation translated through the active transmission interface for at least a subset of the degrees of freedom independently.

As discussed, the system preferably includes a set of output hydraulic lines 136. In some variations, the system may additionally include a set of input hydraulic lines 134. These different legs of hydraulic lines may be substantially similar. Hydraulic lines preferably are of scanner compatible materials in a restricted region. For example, at least the set of output hydraulic lines may be made of MRI compatible materials.

In some variations, the system may include a set of clean break fittings, which function to enable hydraulic lines to be connected and disconnected without pressure loss in the system. The clean break fittings can be integrated into the hydraulic line segments so that they can connect and disconnect on one or both ends. The clean break fittings may be used in proximity to the input or output manipulator systems 110 and 120 so that the manipulators can be changed. The clean break fittings may additionally or alternatively be used in proximity to the active transmission interface 131. In some cases, the components of the system can be reconfigured depending on the intended use of the system. For example, a system with a powered active transmission system can be reconfigured to be a fully passive system by reconfiguring the connections of the hydraulic lines to not include the powered active transmission system.

In some variations, the system may include one or more locking valves integrated into one or more hydraulic line. The locking valve may function as a physical brake to lock hydraulic actuation. There could be locking valves on one or more hydraulic lines so that one or more DoFs may be fixed based on if the locking valve is engaged. In one exemplary implementation, the locking valve is a solenoid valve that when engaged prevents actuation of the hydraulic line. Other types of locking valve mechanisms may alternatively be used. In some variations, a locking valve is a manually engaged mechanism, where a user must physically engage or disengage the valve. In some variations, a locking valve is an electronically controlled device, where the state of the valve can be digitally controlled.

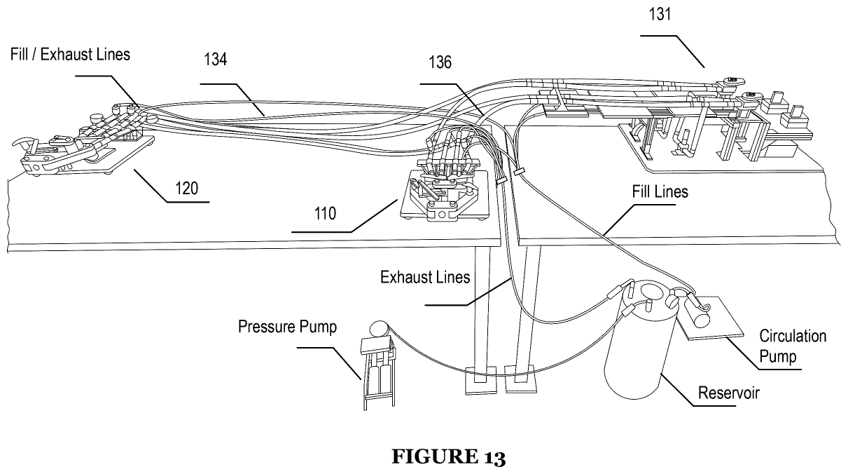

The system may include other hydraulic system sub-components like a reservoir, pressure pump, circulation pump, pressure sensor, fill/exhaust lines and valves, and/or other components as shown in FIG. 13. Fill lines and the exhaust lines may connect to the components of the system like the transmission interfaces, as shown in FIG. 13. In FIG. 13 not all hydraulic/pneumatic connections are shown. Alternative configurations and interconnections may alternatively be used. Similarly, subcomponents such as those described above may include features for use with a hydraulic system. In one variation, the transmission interface can include a fill port, where hydraulic fluid or gas can be added or removed from the system as shown in FIG. 9A and FIG. 9B.

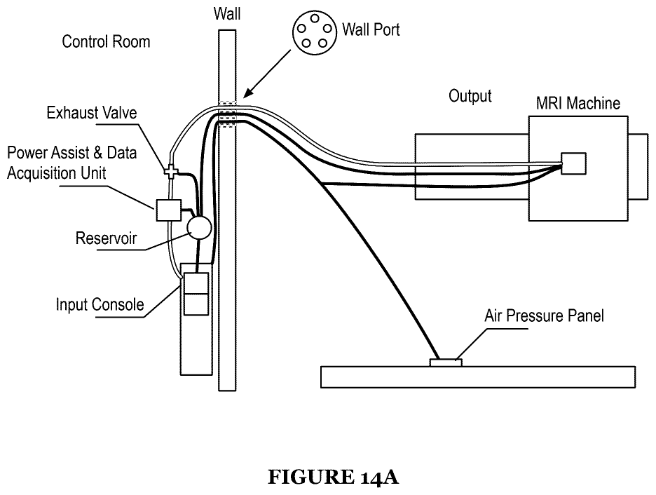

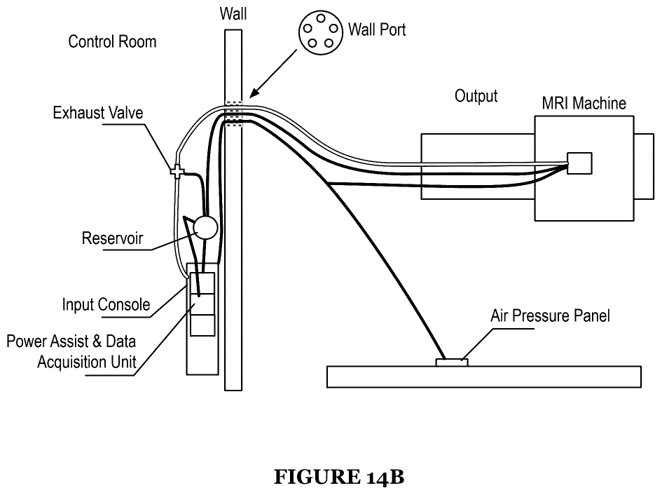

In some variations, a subset of the system components may be arranged on one-side of a physical barrier. As shown in FIGS. 14A and 14B, components like the active transmission interface 131, an input console, hydraulic subsystems (e.g., reservoirs and exhaust valves) may be arranged on a first side of a barrier (e.g., a shielded unrestricted environment), and the output transmission system and output manipulator systems can be arranged on a second side of the barrier (e.g., a restricted environment near a medical scanner). FIG. 14A shows a variation with input and output hydraulic lines. FIG. 14B shows a variation with only output hydraulic lines. This functions to isolate any of the restricted components from the second side (which is where a device like an MRI or CT machine may be used). The input console may include user input device, monitors, visualization system, digital controls, interfaces with external data inputs, and/or other components.

A transmission interface functions to convert between hydraulic actuation and another form of actuation (e.g., mechanical actuation). Transmission interfaces may be used to on the input and output side to translate from hydraulic actuation from a hydraulic line to mechanical actuation of a manipulator system. Transmission interfaces in the case of the active transmission interface 131 may additionally expose a mechanism by which powered actuation and/or sensing may be applied.

As shown in FIG. 6 and FIG. 8, transmission interfaces (active and non-active) and hydraulic lines may be connected in series for one degree of freedom of actuation. Multiple parallel channels may be established and used for controlling multiple DoF. In some instances, the transmission interface used for the input (135) and/or output (137) may be designed with a mechanism in a similar manner to the active transmission interface 131 described here and may be implemented without sensing or powered actuation.

The transmission interface may use two integrated hydraulic-to-mechanical transmission components such that hydraulic actuation and mechanical actuation are linked. In one variation, a transmission interface, include two coupled hydraulic-to-mechanical mechanisms such that, as a whole, the active transmission interface 131 translates from hydraulic actuation to mechanical actuation and back to hydraulic actuation.

In one variation, a transmission interface uses a hydraulic actuator with rolling diaphragm to translate external motion (e.g., linear actuation) to hydraulic actuation within a hydraulic line. Use of a rolling diaphragm may experience reduced friction and smoother actuation. Alternatively, other types of mechanisms may be used to translate external actuation to hydraulic actuation. In one variation, a transmission interface used for the input and/or output manipulator systems can have a rotational actuator (e.g., gear, cable, belt) engage with two linear gears moving in opposing/inverse directions to create inverse hydraulic actuation in two hydraulic lines. A gearing system may be used to alter the translation of hydraulic actuation through the transmission interface.

As shown in FIG. 9A, a transmission line with an input transmission interface 135, an intermediary active transmission interface 131, and an output transmission interface 137 with similar mechanical designs can be connected to form a full hydraulic line/channel. When in a passive mode, actuation at the input or output will translate to the opposing end. When in a controlled augmented mode, a motor of a powered actuation system 132 attached or otherwise coupled to a shaft of a rotational element of the transmission system (as shown in the example of FIG. 9A) may be used to augment actuation. As discussed, an alternative topology may be used whereby the system can use one inactive transmission interface for coupling to a manipulator and an active transmission system for perturbing actuation. As shown in FIG. 9B, a transmission line may include: an active transmission interface 131 coupled mechanically to an input, an output transmission interface 136, and output hydraulic lines integrating the two transmission interfaces.

As the output transmission interface 137 may be used in a restricted region, in some variations, the transmission interfaces or at least the output transmission interface 137 may be made of compatible materials.

The active transmission interface 131 functions to supplement and/or augment the hydraulic actuation between the input manipulator 110 and the output manipulator 120. The active transmission interface 131 allows sensing and/or actuation augmentation to be used within the hydraulic transmission system 130.

The active transmission interface 131 is an intermediary component that is serially integrated between the input manipulator system 110 and the output manipulator system 120. The active transmission interface 131 may include or be connect to a powered actuation system 132 such that the active transmission interface 131 is a powered active transmission interface 131. Additionally or alternatively, the active transmission interface 131 may include a sensing system 133 such that the active transmission interface 131 is a sensor-enabled active transmission interface 131. The powered actuation system 132 and sensing system 133 may be used in combination to make a sensor-enabled and powered active transmission interface 131.

As in the non-active transmission interfaces, the active transmission interface 131, in one variation, may include two coupled hydraulic-to-mechanical mechanisms such that, as a whole, translates from hydraulic actuation on one interface (e.g., an input) to mechanical actuation and back to hydraulic actuation at another interface (an output). The mechanical actuation portion may be augmented with a powered actuation system and/or be sensed with a sensing system.

In one variation, the two hydraulic-to-mechanical mechanisms are mechanically coupled together. In one example, the exemplary hydraulic-to-mechanical mechanisms can both engage with a rotary gear such that their actuation is coupled. A motor mechanically coupled to the rotary gear could be actively engaged to amplify or resist actuation. The powered actuation system 132 may have a motor directly or indirectly (e.g., through a gearing system) to a shaft extending from the rotary gear. A sensing system 133 may additionally or alternatively integrate with the rotary gear to sense the actuation transferred through the hydraulic transmission system 130. For example, the rotation of the gear may be sensed using an encoder or other sensing mechanism.

Other mechanisms may alternatively be used to function as a hydraulic transmission interface with integrated powered actuation and/or sensing. In one exemplary variation, an alternative transmission mechanism may be used so that two hydraulic-to-mechanical transmissions could be physically disconnected where an electronic or other control mechanism is used to sense and/or control the mechanical actuation of each portion of the interface. This may be used so that all or part of the system could be remotely controlled.

Alternatively, if used on the input transmission interface, then the active transmission interface may translate mechanical actuation of the input manipulator to hydraulic actuation.

The powered actuation system 132 functions to augment the actuation occurring within the hydraulic transmission system 130. In one variation, the powered actuation system 132 includes a motor that mechanically couples to a hydraulic-to-mechanical transmission mechanism. The motor may couple to a rotary gear of a hydraulic-to-mechanical transmission through a motor attachment shaft and/or using a gear train. The powered actuation system 132 may use a backdrivable motor that couples with a rotary gear of the hydraulic-to-mechanical transmission mechanism such that it can allow rotation without being driven. The powered actuation system 132 may include other components used in the operation and control of a motor-powered system such as motor encoders, motor drivers, power systems, control systems, and/or other suitable components.

The powered actuation system 132 may include one motor for each channel and controlled DoF. Each motor can be independently controlled.

The powered actuation system is preferably controlled by a control system 140. The control system may manage how actuation is translated through the system. The powered actuation system 132 may be used to provide resistive forces or supportive forces. The powered actuation system 132 can additionally disengage to allow actuation to occur passively. The powered actuation system may be controlled based on actuation from the input manipulator system 110 and/or the output transmission interface 137, and optionally from input from the sensing system 133, digital control inputs 150 and/or supplemental data input.

The sensing system 133 functions to collect data during use of the device. The sensing system 133 can preferably track the actuation of the system. In one variation, the sensing system 133 can provide data that is used as input to a control system 140 of the powered actuation system 132. In this way, the system can respond to how a user is manipulating the input manipulator system 110 and/or the haptic feedback from the output manipulator system 120. In another variation, actuation data may be collected in coordination with medical data from the same time. This can be used to train data models used to automate or otherwise augment actuation in later uses of the system. In some alternative variations, a sensor enabled active transmission interface 131 may be used independent of powered actuation. This non-powered actuation implementation may be a selectable mode if a fully passive mode is used but sensing and data collection is still desired.

The sensing system 133 may use encoders, pressure sensors, and/or other sensors to monitor state of the system that can be used for determining how to control the powered actuation system 132. In one variation, the sensing system 133 may be integrated into the active transmission interface 131. For example, the sensing system may sense mechanical actuation of a transmission interface. In one exemplary variation, an encoder coupled to a rotary gear of a hydraulic transmission interface may be used to track actuation applied between two coupled hydraulic lines.

In another variation, a sensing system 133 may be integrated into another part of the system or even be data provided by an outside system. For example, a sensing system 133 could be hydraulic sensing system integrated into some part of the hydraulic transmission system 130. This may include measuring pressure and/or actuation within a hydraulic line. In one variation, pressure may be measured on two legs of the hydraulic transmission system 130—measuring pressure from an input hydraulic line and from an output hydraulic line. A pressure differential calculated from the sensed pressures may be used to do friction compensation and/or inertia compensation. Other sensors may alternatively be used.

The system may additionally or alternatively include one or more additional data input 160, which functions as a supplemental data source that can be used within the system. The data input 160 can additionally or alternatively be used to control a powered actuation system 132. Such a supplemental data input 160 may be a medical data input 160. For example, data from an active scanner device (e.g., an MRI machine or CT scanner) could be used to dynamically control, at least in part, the powered actuation system 132.

The powered actuation system 132 may be used in a variety of ways. In one variation, the powered actuation system 132 may be used in one of a set of modes such as a passive mode, power-assist mode, and/or a fully autonomous mode. The powered actuation system 132 could dynamically switch between such modes or alternatively be configured to always operate in one of the described modes.

A control system 140 functions to manage augmentation of actuation. The control system can include configuration or otherwise be configured to control the powered actuation system to at least partially augment hydraulic actuation (e.g., the haptic interactions) translated through the active transmission interface 131 between the input manipulator system and the output manipulator system. The control system 140 may be communicatively coupled to and integrated with the powered actuation system 132, such that the control system 140 can manage augmentation of actuation applied by the powered actuation system 132 to the active transmission interface 131.

The control system 140 may include one or more processors and one or more computer-readable mediums (e.g., non-transitory computer-readable mediums) storing instructions that, when executed by the one or more processors, cause the control system 140 to perform operations related to controlling actuation translated through the active transmission interface 131. The control system 140 may alternatively be configured as a hardware implementation where the control system 140 includes an electronic control system. Both variations may include configuration or be configured to control the powered actuation system to at least partially augment hydraulic actuation translated through the active transmission interface between the input manipulator system and the output manipulator system. The operations may vary depending on the implementation and state.

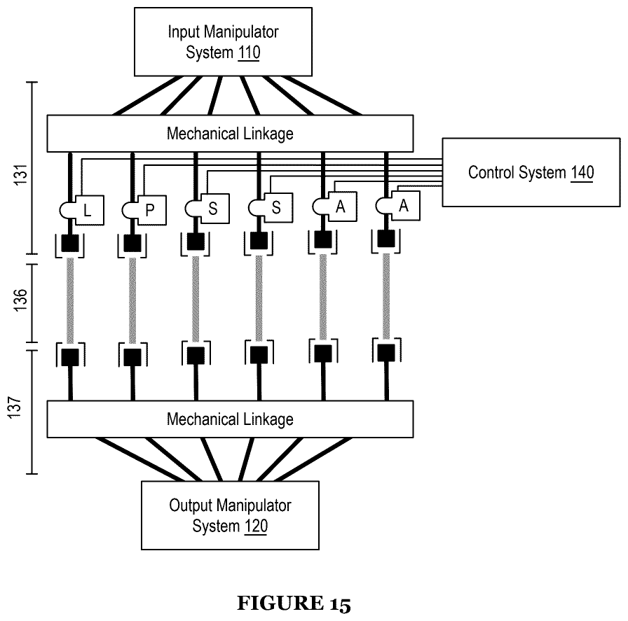

In one variation, the control system 140 may include configuration to translate hydraulic actuation through the active transmission interface 131 in one of a set of modes, where at least one mode of the set of modes is a mode that augments hydraulic actuation. This may include different distinct modes or dynamic modes that adjust augmentation in real-time. The various modes may include a passive mode, a power-assist mode, and/or an automated mode. Different degrees of freedom may be individually controlled with dynamic control. As shown in FIG. 15, a system with six DoF may have the different DoF in different states where the powered actuation system 132 is controlled such that: a first motor is in a locked state (L), a second motor is in a passive state (P), a third and fourth motor are in power-assist or semi-automated mode(S), and a fifth and sixth motors are in an automated state (A). This establishes the different channels in a locked state, passive state, semi-automated state (e.g., a power assist mode) or in an automated state. Additionally, the set of different channels may be controlled in a coordinated manner.

In a passive mode, the powered actuation system 132 may be inactive and rotate freely such that the output actuation is a direct result of input actuation. When configured to apply a passive mode for one or more DoF, the control system 140 may deactivate a motor of a relevant transmission interface 131. Accordingly, in a passive mode, the powered actuation system 132 (and more specifically a motor of the powered actuation system 132) can be unpowered/unenergized. The motor of the powered actuation system 132 may remain mechanically engaged and coupled to the active transmission interface, but may not apply powered actuation. The motor of the powered actuation system 132 can be backdrivable so that it can be rotated freely by user control at the input 110.

In an alternative implementation, when in a passive mode, a motor of the powered actuation system 132 may be mechanically disengaged from the active transmission interface 131. Being mechanically disengaged may mean that the motor is mechanically decoupled and unconnected from some mechanism to drive or intervene with actuation. A clutch may be used to facilitate mechanical disengagement. Use of a clutch in a passive mode, may allow the motors to potentially expand viable motor boxes such as non-backdrivable motors.

In a power-assist mode (i.e., a “semi-automated” mode), the powered actuation system 132 may intervene, augment, or otherwise alter actuation supplied by the input. When in a power-assist mode, hydraulic actuation input from the input manipulation system is translated by a function to hydraulic actuation output delivered to the output manipulation system. In this way actuation is the combination of actuation or force at the input manipulator system 110 and/or the output manipulator system 120 with the actuation force from the powered actuation system 132 at the active transmission interface 131.

A power-assist mode may be implemented with one or more different features to assist or otherwise alter the actuation control and/or haptic feedback. A power assist mode may be used in various ways. In some examples power assist may be used to filter or otherwise adjust input actuation, compensate for various aspects of operation, and/or alter or enhance haptic feedback.

In a power assist mode variation for filtering input, a power-assist mode may be used for noise removal or signal smoothing. This may, for example, be used to remove a user's input tremors from transferring to the output. A lowpass filter on the actuation may be used to prevent sudden jerks of motion.

In a power assist mode variation for compensation, the powered actuation system 132 may be used to compensate for friction and/or inertia within the system. This can be used to remove the appearance of friction or inertia to the operator of the systems. Configuration may include sensing friction or inertia and then applying counteractive control to the powered actuation system 132 to counter the friction or inertia. Sensing friction or inertia may include sensing pressure differentials between an input transmission line and an output transmission line.

In a power assist mode for haptic feedback, configuration controls the powered actuation system 132 to alter haptic feedback experienced by the input manipulator system. Haptic feedback augmentation may be used to amplify (or alternatively deamplify) haptic feedback transferred from the output back to the input. In some variations, such haptic feedback augmentation could be based on externally sensed input(s). Sensed inputs may be based on the sensed state of the subject, sensed state of the operator, and/or sensed state from an imaging device. For example, the position of an operating tool in relation to a region of interest in the patient, as determined through MRI imaging or other scanner data, could be used to artificially supply haptic feedback to the operator.

In another variation, other input effects could be implemented through dynamic control of the power assist mode. For example, added or removed hysteresis in movement resistance could be enabled by the powered actuation system 132.

In a fully autonomous mode, the powered actuation system 132 may operate without input supplied by the input manipulator system 110. In other words, in an automated mode, hydraulic actuation may be driven entirely by the intermediary active transmission 131 using the powered actuation system 130. The fully autonomous mode may be used for fully autonomous procedures or steps. Alternatively, the autonomous mode may be used for select actions. For example, an autonomous mode may be used to move an output manipulator (e.g., an operating tool) into position before enabling manual control. In another example, a subset of DoF may be in an autonomous mode. For example, a doctor may move a tool into a position and then lock the position of tool relative to the medical imaging. The tool may track the position of the tool relative to the medical imaging so that it stays aligned to the point of the body. In another example, select actions may be performed using an autonomous mode. Such autonomous modes may be controlled based on historical data collected through previous operation of similar systems.

### 3. Method

A method for a hydraulic transmission system of a teleoperation device with haptic feedback can include controlling a system such as described above to provided augmented actuation. The method functions to use a hydraulic transmission to alter hydraulic actuation and feedback. The method is preferably implemented through the operation of a hydraulic manipulator. The method is preferably used in combination with a system variation as described herein but may alternatively be used with alternative system implementations.

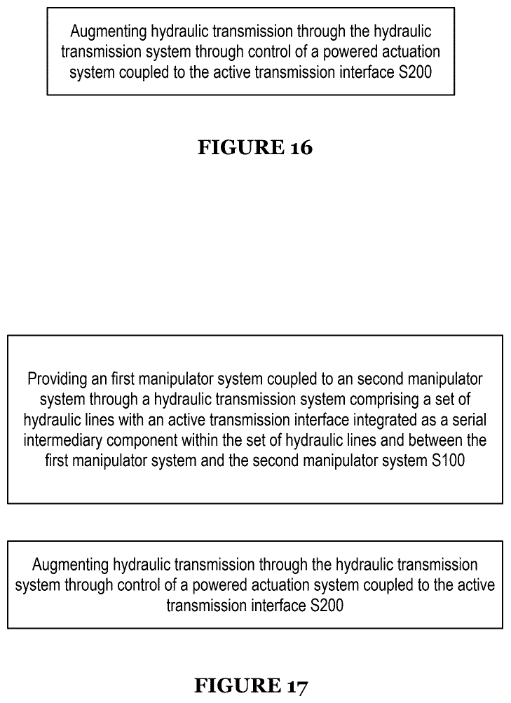

The method may include, at a system such as described above with a hydraulic transmission system with an active transmission interface as an intermediary component between an input manipulator system and an output manipulator system, augmenting hydraulic transmission through the hydraulic transmission system through control of a powered actuation system coupled to the active transmission interface S200 as shown in FIG. 16.

The method may additionally include assembling, configuring, or otherwise providing the system. Accordingly, as shown in FIG. 17, the system may include providing an first manipulator system coupled to an second manipulator system through a hydraulic transmission system comprising a set of hydraulic lines with an active transmission interface integrated as a serial intermediary component within the set of hydraulic lines and between the first manipulator system and the second manipulator system S100, and augmenting hydraulic transmission through the hydraulic transmission system through control of a powered actuation system coupled to the active transmission interface S200. The first manipulator system may be an input manipulator system and the second manipulator system may be an output manipulator system. The method may additionally or alternatively be used to transfer from an output device to an input device as such the first manipulator system may be an output manipulator system and the second manipulator system may be an input manipulator system.

In some variations, the method may be implemented in connection with a non-transitory computer-readable medium storing instructions that, when executed by one or more computer processors of a computing platform, cause the computing platform to perform operations comprising: providing an input manipulator system coupled to an output manipulator system through a hydraulic transmission system comprising a set of hydraulic lines with an active transmission interface integrated as a serial intermediary component within the set of hydraulic lines and between the input manipulator system and the output manipulator system S100, and augmenting hydraulic transmission through the hydraulic transmission system through control of a powered actuation system coupled to the active transmission interface S200. The computer-readable medium may additionally include configuration for any suitable variation described herein.

In a similar way, the method may be implemented as part of a system such as a control system whereby the system includes one or more computer-readable mediums storing instructions that, when executed by the one or more computer processors, cause a computing platform to perform operations comprising: augmenting hydraulic transmission through the hydraulic transmission system through control of a powered actuation system coupled to the active transmission interface S200. The system may additionally include the components of the system described herein.

Block S100 functions to provide, assemble, configure, or otherwise perform operations in connection with a system as described herein. In some variations, the method may be performed with a system differing in some manner from the system described herein.

Block S100 may specifically include providing an input manipulator system coupled to an output manipulator system through a hydraulic transmission system comprising a set of hydraulic lines with an active transmission interface integrated as a serial intermediary component within the set of hydraulic lines and between the input manipulator system and the output manipulator system. The providing the system may include the producing or setting up of any of the components described herein such as input manipulator system(s), output manipulator system(s), a hydraulic transmission system, an active transmission interface, a powered actuation system, a sensing system, input hydraulic lines, input transmission interface, output hydraulic lines, output transmission interface, control system, a digital control input, and/or a data input.

In these method variations, the method may be applied with a system including any suitable hydraulic network topology.

In one variation, the method may be used with a hydraulic transmission system that includes a single output leg of hydraulic lines bridging a distance between the active transmission interface and an output transmission interface. In this variation, the input manipulator system may be directly coupled to the input of the active transmission interface through a linkage system; and translating the actuation input to actuation at the input of the active transmission interface may include translating the actuation input through mechanical actuation of the linkage system.

In another variation, the method may be applied with a hydraulic transmission system that includes an input leg and output leg of hydraulic lines. The input hydraulic lines bridge a distance between the active transmission interface and an input transmission interface. The output hydraulic lines bridge a distance between the active transmission interface and an output transmission interface. In this variation, the input manipulator system is coupled to the input of the active transmission interface through a set of input hydraulic lines of the set of hydraulic lines; and translating the actuation input to actuation at the input of the active transmission interface includes translating the actuation input from the input manipulator system through the set of input hydraulic lines as hydraulic actuation to active transmission interface.

Block S200, which includes augmenting hydraulic transmission through the hydraulic transmission system through control of a powered actuation system coupled to the active transmission interface, functions to control how actuation, force, and/or pressure is conveyed within the hydraulic transmission system.

Augmenting hydraulic transmission through the hydraulic transmission system is used to dynamically adjust how an input to an input manipulator and/or an output at an output manipulator is experienced and/or expressed. In some variations, augmenting hydraulic transmission through the hydraulic transmission system may include affecting manipulation at the input manipulator system, affecting manipulation at the output manipulator system, translating manipulation at the input and the output manipulator systems through a hydraulic system that is configured to have an active transmission interface as a serial intermediary component between the input manipulator system and the output manipulator system, and augmenting hydraulic transmission through the active hydraulic transmission system by controlling a powered actuation system coupled to the active transmission interface.

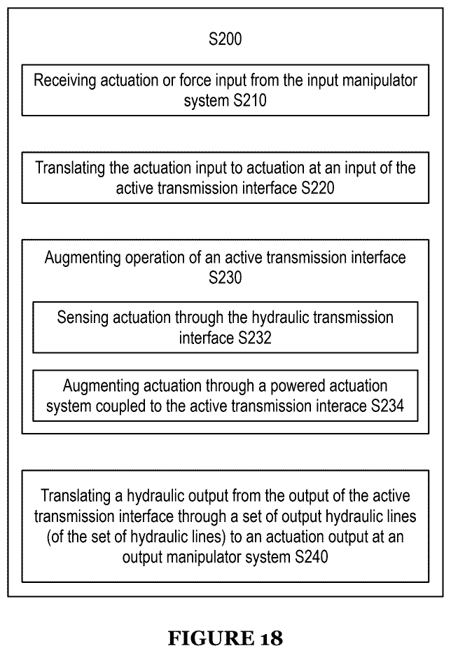

When described how it may be expressed when a user applies some input, augmenting hydraulic transmission through the hydraulic transmission system may include, as shown in FIG. 18: receiving actuation or force input from the input manipulator system S210; translating the actuation input to actuation at an input of the active transmission interface S220; augmenting operation of a hydraulic transmission system S230; and translating a hydraulic output from the output of the active transmission interface through a set of output hydraulic lines (of the set of hydraulic lines) to an actuation output at an output manipulator system S240.

Block S210, which includes receiving actuation or force input from the input manipulator system, functions to receive input of an operator. The input is preferably implemented through mechanical manipulation of some mechanism with a number of mechanical DoFs. Haptic feedback is preferably also experienced at the input manipulator. In some cases, other input mechanisms may be used in the input device.

In one variation, a single input manipulator is designed to receive the manual input of one operator. In other variations, multiple input manipulators may be used to deliver multiple inputs. For example, for certain types of operations, multiple operator tools or tools with multiple controllable elements may be used. Multiple different operators (e.g., doctors) may supply input to control a subset of the elements. In some variations, one or more aspects may be controlled by an automated system.

In addition to receiving input from a manipulator system, the method may additionally receive input from a digital control input or another data input. A digital control input may be some input signal that is supplied from digital or electronic device. In one example, the method may be able to additionally handle receiving an input signal from a digital device over a network. This may function to allow teleoperation from practitioners far removed. For example, when used for a teleoperation, an expert practitioner could connect in to handle some aspects of an operation from a remote location (e.g., connected via a communication network), but a local practitioner could still use the input manipulator to have direct actuation control to assist or even take over should something happen with the remote connection.

Block S220, which includes translating the actuation input to actuation at an input of the active transmission interface, functions to relay the input from the input manipulator to a hydraulic transmission system. The hydraulic system transfers the input to actuation at an output. This transfer of input may or may not include some augmentation at given point of time. The use of a hydraulic system may be used so that the device can be used in environments where electronics and/or metal components are not feasible such as within an MRI device.

Block S240, which includes translating a hydraulic output from the output of the active transmission interface through a set of output hydraulic lines to an actuation output at an output manipulator system, functions to convey some output over a hydraulic line to an output manipulator system. This conveyed output is used to control execution of some action (e.g., motion or applied force) at some output mechanism. In some applications, an output manipulator may be situated in an environment where electronics and/or metal components may not be used. As such a portion of the hydraulic line to the output manipulator and the output manipulator may be made of compatible materials and components. For example, when used with an MRI device, the output manipulator may be made of plastic and non-metallic materials.

The output manipulator may mirror that of the input manipulator. However, the output manipulator may be of a different design and the output may be of a different actionable form from the input. For example, an angular DoF at the input may be used to control a linear DoF at the output.

In one variation, an input manipulator may be directly mechanically coupled to an interface of the active transmission interface. In another variation, the input manipulator may be indirectly coupled to the active transmission interface through a hydraulic connection (e.g., input hydraulic lines).

The method can support reconfiguration of the system to alter forms of actuation control. In one variation, the method can include reconfiguring one or more DoFs for passive control and/or augmented or autonomous control. For example, the hydraulic lines connections can be configured to route around an active transmission interface to be used in a passive mode. Similarly, the hydraulic lines can be configured to use different types of input and/or output manipulators. In another variation of reconfiguration, hydraulic brakes may be engaged or disengaged to selectively enable or disable select DoF.

Block S230, which includes augmenting operation of a hydraulic transmission system, functions to collect information on operation and/or to alter the actuation, force or pressure transmitted between the input and outputs of the hydraulic transmission system. This may be used to enable powered actuation and/or sensing and monitoring of actuation.

In one variation, augmenting the hydraulic actuation may additionally or alternatively include sensing actuation through the hydraulic transmission interface S232.

Additionally or alternatively, augmenting the hydraulic actuation in one variation includes augmenting actuation through a powered actuation system coupled to the active transmission interface S234. Augmenting the hydraulic actuation/force through the powered actuation system S234 may more specifically include dynamically controlling the powered actuation system coupled to the active transmission interface as actuation/force is translated between the input of the active transmission interface to an output of the active transmission interface.

Augmenting actuation/force through a powered actuation system S234 may include augmenting actuation transmitted through the hydraulic system in one of a set of modes. Exemplary modes may include a passive mode, power-assist mode, and/or an automated mode. Augmenting actuation/force through a powered actuation system may additionally include switching mode for one or more channels (DoF) of the hydraulic transmission system. Additionally, dynamically controlling the powered actuation system may additionally include dynamically controlling the powered actuation system independently for different subsets of channels. Each channel may include a line of transmission interfaces and hydraulic lines coupling to a degree of freedom of an input and output manipulator.

A passive mode functions to enable the system to operate, at least temporarily, without active augmentation. When in a passive mode, the hydraulic transmission system will generally function as hydraulic system to transmit actuation/force from an input to an output. A passive mode may be engaged for all DoF (thereby for each line of the hydraulic transmission system). A passive mode may alternatively be engaged for a subset of DoF (e.g., for one or more of the lines of the hydraulic transmission system). In a passive mode, the method may include transmitting actuation through the hydraulic system without augmentation. Augmenting the hydraulic actuation/force through an active hydraulic transmission interface may include, at a control system, disengaging or deactivating a motor of the powered actuation system coupled to an active transmission interface for each line set to a passive mode. If the full hydraulic transmission system is set to a passive state, then augmenting hydraulic actuation/force when in a passive mode can include disengage or deactivating each motor of the powered actuation system coupled to the active transmission interface.

A power-assist mode functions to alter actuation supplied by some input. The input supplied by an operator to the input manipulator can be altered by the powered actuation system when in a power-assist mode.

This may be used to alter the output actuation. This may additionally or alternatively be used to alter the haptic feedback experienced by the input manipulator. As discussed herein, power-assist modes can be used for various power-assist features such as reducing effects of friction or inertia, smoothing or filtering input, and/or altering actuation in other ways.

In some variations, the power-assist mode may be used in compensating for losses or deficiencies in the system. This may be used to counteract friction or inertia for example. In one variation, differential pressure sensing performed across the hydraulic lines as shown in FIGS. 9A and 9B, can be used to apply friction compensation and/or inertia compensation using the powered actuation system. In this variation, augmenting actuation/force through a powered actuation system can include sensing pressure differential and determining friction and/or inertia and applying, using the powered actuation system, a compensating force based on the friction or inertia.

In some variations, the power-assist mode may be used for filtering or changing how an input is translated to an output. In such a mode, augmenting actuation/force through a powered actuation system may include applying a filter to actuation/force from an input manipulator system. The filter could be a lowpass filter to eliminate sudden movements, jerk motions, and/or tremors. This may smooth out actuation at the output manipulator. Alternatively, any suitable transform or function may be used to translate an input force/actuation to an output force/actuation.

In some variations, the power assist mode may be used to alter input based one or more additional data input. The additional data input may be sensed data. The additional data input may additionally or alternatively be a medical data input. As mentioned, the method may include receiving an additional data input, and wherein augmenting actuation through a powered actuation system may include controlling input-output translation based on additional data input(s). An example of a medical data input may include scanner data from an MRI, CT scanner, heart rate, respiratory rate, and/or any suitable data related to medical conditions or conditions of a subject.

In some variations, the additional data input provides information on sensed or detected state of the output device. This may enable MRI imaging or other forms of monitoring to alter the controls of a device.

As the system enables use of a manipulator during live scanning with restrictive scanning regions, the method may enable novel operations using live real-time scanning data. For example, augmenting control may be used for controlling actions of an output manipulator system during live MRI scanning, wherein controlling the actions is based in part on MRI images resulting from the scanning.

For example, using MRI imaging, the powered active transmission interface may alter the power-assist mode based on where a tool is in relation to a particular region of the body. This may be used to prevent moving a tool into restricted regions, to alter the precision of the tool when in vicinity to a targeted region, and/or enable other operation features.

As another variation, a power assist mode may be used to enhance or generate haptic feedback. in a power assist mode used to enhance haptic feedback, augmenting actuation/force through the powered actuation system may include, at a powered actuation system, triggering a haptic feedback action to at least an input, wherein triggering is based on a detected condition. The detected condition may depend on additional data inputs. The haptic feedback may simulate a synthetic wall, barrier, region with “rough” feeling actuation (e.g., increased friction), a region with smooth actuation (e.g., decreased friction), vibration alerts, and/or other forms of haptic feedback.

An automated mode functions to control actuation entirely or primarily using the powered actuation system 132. An autonomous mode may be engaged for one or more degrees of freedom. In a fully automated mode, actuation is fully driven by the automated control of the powered actuation system. In some variations, a partial automated mode may enable input supplied from the input manipulator system to dynamically adjust or take over from the automated mode. For example, an automated mode may be engaged. For a time, a practitioner may let the automated mode perform its task, but if desired, the practitioner could begin to supply input to the input manipulator system and that could override the automated control while input is supplied. In some variations, input to the input manipulator system may deactivate the automated mode. In some variations, input will adjust the automated actions, but then automated control can restart immediately.

### 4. System Architecture

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, but any suitable dedicated hardware device can (alternatively or additionally) execute the instructions.

In one variation, a system comprising of one or more computer-readable mediums (e.g., non-transitory computer-readable mediums) storing instructions that, when executed by the one or more computer processors, cause a computing platform to perform operations comprising those of the system or method described herein such as: providing an input manipulator system coupled to an output manipulator system through a hydraulic transmission system comprising a set of hydraulic lines with an active transmission interface integrated as a serial intermediary component within the set of hydraulic lines and between the input manipulator system and the output manipulator system, and augmenting hydraulic transmission through the hydraulic transmission system through control of a powered actuation system coupled to the active transmission interface.

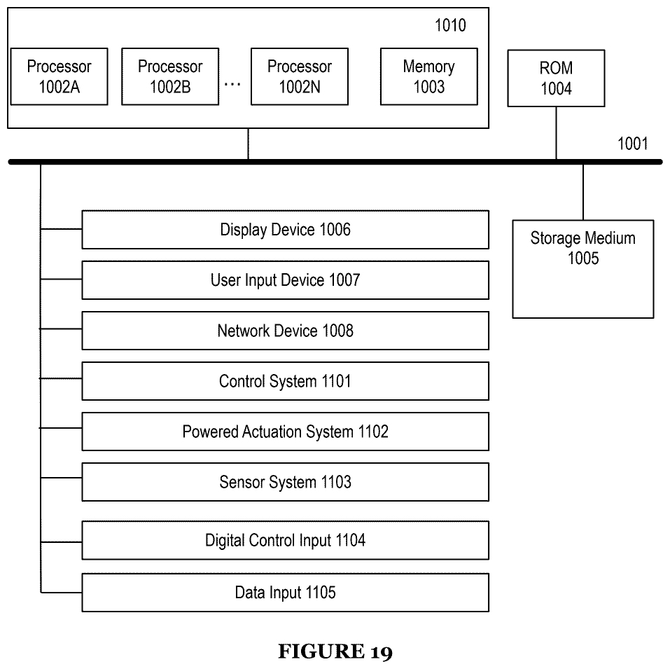

FIG. 19 is an exemplary computer architecture diagram of one implementation of the system. In some implementations, the system is implemented in a plurality of devices in communication over a communication channel and/or network. In some implementations, the elements of the system are implemented in separate computing devices. In some implementations, two or more of the system elements are implemented in same devices. The system and portions of the system may be integrated into a computing device or system that can serve as or within the system.

The communication channel 1001 interfaces with the processors 1002A-1002N, the memory (e.g., a random-access memory (RAM)) 1003, a read only memory (ROM) 1004, a processor-readable storage medium 1005, a display device 1006, a user input device 1007, and a network device 1008. As shown, the computer infrastructure may be used in connecting control system 1101, powered actuation system 1102, sensor system 1103, digital control input 1104, data input 1105, and/or other suitable computing devices.

The processors 1002A-1002N may take many forms, such CPUs (Central Processing Units), GPUs (Graphical Processing Units), microprocessors, ML/DL (Machine Learning/Deep Learning) processing units such as a Tensor Processing Unit, FPGA (Field Programmable Gate Arrays, custom processors, and/or any suitable type of processor.

The processors 1002A-1002N and the main memory 1003 (or some sub-combination) can form a processing unit 1010. In some embodiments, the processing unit includes one or more processors communicatively coupled to one or more of a RAM, ROM, and machine-readable storage medium; the one or more processors of the processing unit receive instructions stored by the one or more of a RAM, ROM, and machine-readable storage medium via a bus; and the one or more processors execute the received instructions. In some embodiments, the processing unit is an ASIC (Application-Specific Integrated Circuit). In some embodiments, the processing unit is a SoC (System-on-Chip). In some embodiments, the processing unit includes one or more of the elements of the system.

A network device 1008 may provide one or more wired or wireless interfaces for exchanging data and commands between the system and/or other devices, such as devices of external systems. Such wired and wireless interfaces include, for example, a universal serial bus (USB) interface, Bluetooth interface, Wi-Fi interface, Ethernet interface, near field communication (NFC) interface, and the like.

Computer and/or Machine-readable executable instructions comprising of configuration for software programs (such as an operating system, application programs, and device drivers) can be stored in the memory 1003 from the processor-readable storage medium 1005, the ROM 1004 or any other data storage system.

When executed by one or more computer processors, the respective machine-executable instructions may be accessed by at least one of processors 1002A-1002N (of a processing unit 1010) via the communication channel 1001, and then executed by at least one of processors 1001A-1001N. Data, databases, data records or other stored forms data created or used by the software programs can also be stored in the memory 1003, and such data is accessed by at least one of processors 1002A-1002N during execution of the machine-executable instructions of the software programs.

The processor-readable storage medium 1005 is one of (or a combination of two or more of) a hard drive, a flash drive, a DVD, a CD, an optical disk, a floppy disk, a flash storage, a solid-state drive, a ROM, an EEPROM, an electronic circuit, a semiconductor memory device, and the like. The processor-readable storage medium 1005 can include an operating system, software programs, device drivers, and/or other suitable sub-systems or software.

As used herein, first, second, third, etc. are used to characterize and distinguish various elements, components, regions, layers and/or sections. These elements, components, regions, layers and/or sections should not be limited by these terms. Use of numerical terms may be used to distinguish one element, component, region, layer and/or section from another element, component, region, layer and/or section. Use of such numerical terms does not imply a sequence or order unless clearly indicated by the context. Such numerical references may be used interchangeable without departing from the teaching of the embodiments and variations herein.

As a person skilled in the art will recognize from the previous detailed description and from the figures and claims, modifications and changes can be made to the embodiments of the invention without departing from the scope of this invention as defined in the following claims.

We claim:

1. A system for remote operation of a device within a medical scanner comprising:
an input manipulator system situated in a shielded region outside of the medical scanner;
an output manipulator system situated within a scanning region of the medical scanner;
a hydraulic transmission system comprising:
an intermediary active transmission interface serially integrated between the input manipulator system and the output manipulator system and within the shielded region, the active transmission interface comprising a powered actuation system wherein force applied to the input manipulator system or the output manipulator is mechanically transferred through the active transmission interface with optional intervention by the powered actuation system,
an output transmission interface that is coupled to the output manipulator system, and
a set of output hydraulic lines connecting to the intermediary active transmission interface in the shielded region to the output transmission interface in the scanning region.

2. A system comprising:
an input manipulator system;
an output manipulator system;
a hydraulic transmission system comprising:
an intermediary active transmission interface integrated between the input manipulator system and the output manipulator system, the intermediary active transmission interface comprising a powered actuation system,
an output transmission interface that is coupled to the output manipulator system, and
a set of output hydraulic lines that connects on one end to the active transmission interface and on the opposing end to the output transmission interface.

3. The system of claim 2, further comprising a control system comprising configuration to control the powered actuation system to at least partially augment hydraulic actuation translated through the active transmission interface between the input manipulator system and the output manipulator system.

4. The system of claim 3, wherein configuration to control the powered actuation system to augment hydraulic actuation comprises configuration to translate hydraulic actuation through the active transmission interface in one of a set of modes, where at least one mode of the set of modes is a mode that augments hydraulic actuation.

5. The system of claim 4, wherein the set of modes comprises a passive mode, where hydraulic actuation is transferred through the active transmission interface without active augmentation.

6. The system of claim 4, wherein the set of modes comprises a power assist mode, whereby hydraulic actuation input from the input manipulation system is translated by a function to hydraulic actuation output delivered to the output manipulation system.

7. The system of claim 4, wherein the set of modes comprises a mode that alters haptic feedback experienced by the input manipulator system.

8. The system of claim 4, wherein the set of modes comprises an automated mode whereby hydraulic actuation is driven entirely by the intermediary active transmission using the powered actuation system.

9. The system of claim 3, wherein the output manipulator system and the input manipulator system comprise corresponding degrees of freedom with an output hydraulic line of the set of output hydraulic lines for each degree of freedom; and wherein the control system can control the powered actuation system to independently augment hydraulic actuation translated through the active transmission interface for at least a subset of the degrees of freedom.

10. The system of claim 3, further comprising a medical data input, wherein the control system controls the powered actuation system at least partially based on the medical data input.

11. The system of claim 2, wherein the output manipulator system is made of material compatible with a magnetic resonance imaging (MRI) machine.

12. The system of claim 2, wherein the input manipulator system and the intermediary active transmission interface is positioned within a shielded environment, and wherein the output manipulator system, and at least part of the set of output hydraulic lines are in a scanning region of a medical scanning device.

13. The system of claim 2, further comprising an additional digital control input, wherein output of the intermediary active transmission interface is based on a combination of input originating from the input manipulator system and the additional digital control input.

14. The system of claim 2, wherein the input manipulator system is directly mechanically coupled to an input interface of the intermediary active transmission interface.

15. The system of claim 2, wherein the hydraulic transmission system further comprises an input transmission interface that is coupled to the input manipulator system and a set of input hydraulic lines that connect on one end to the active transmission interface and on the opposing end to the input transmission interface.

16. The system of claim 1, further comprising a sensing system coupled to the active transmission interface and configured to measure actuation transmitted through the hydraulic transmission system.

17. A method comprising:
constructing a first manipulator system coupled to an second manipulator system through a hydraulic transmission system comprising a set of hydraulic lines with an active transmission interface integrated as a serial intermediary component within the set of hydraulic lines and between the first manipulator system and the second manipulator system,
augmenting hydraulic transmission through the hydraulic transmission system through control of a powered actuation system coupled to the active transmission interface.

18. The method of claim 17, wherein augmenting hydraulic transmission comprises:
receiving actuation input from the first manipulator system;
translating the actuation input to actuation at an input of the active transmission interface;
dynamically controlling a powered actuation system coupled to the active transmission interface as actuation is translated from the input of the active transmission interface to an output of the active transmission interface,
translating a hydraulic output from the output of the active transmission interface through a set of output hydraulic lines of the set of hydraulic lines to actuation output at an output manipulator system.

19. The method of claim 18, wherein the input manipulator system is directly coupled to the input of the active transmission interface through a linkage system; and wherein translating the actuation input to actuation at the input of the active transmission interface comprises translating the actuation input through mechanical actuation of the linkage system.

20. The method of claim 18, wherein the first manipulator system is coupled to the input of the active transmission interface through a set of input hydraulic lines of the set of hydraulic lines; and wherein translating the actuation input to actuation at the input of the active transmission interface comprises translating the actuation input from the first manipulator system through the set of input hydraulic lines as hydraulic actuation to active transmission interface.

21. A non-transitory computer-readable medium storing instructions that, when executed by one or more computer processors of a computing platform, cause the computing platform to perform operations comprising:
augmenting hydraulic transmission through the hydraulic transmission system through control of a powered actuation system coupled to the active transmission interface, which comprises:
receiving actuation input from the input manipulator system;
translating the actuation input to actuation at an input of the active transmission interface;
dynamically controlling a powered actuation system coupled to the active transmission interface as actuation is translated from the input of the active transmission interface to an output of the active transmission interface, wherein the
translating a hydraulic output from the output of the active transmission interface through a set of output hydraulic lines of the set of hydraulic lines to actuation output at an output manipulator system.

22. A system comprising of:
one or more computer-readable mediums storing instructions that, when executed by the one or more computer processors, cause a computing platform to perform operations comprising:
augmenting hydraulic transmission through the hydraulic transmission system through control of a powered actuation system coupled to the active transmission interface, which comprises:
receiving actuation input from the input manipulator system;
translating the actuation input to actuation at an input of the active transmission interface;
dynamically controlling a powered actuation system coupled to the active transmission interface as actuation is translated from the input of the active transmission interface to an output of the active transmission interface, wherein the
translating a hydraulic output from the output of the active transmission interface through a set of output hydraulic lines of the set of hydraulic lines to actuation output at an output manipulator system.
